SEQUENCE LISTING (6 sequences)

SEQ ID NO: 1 - RNA, 23 nt, human
ucuuugguuaucuagcuguauga

SEQ ID NO: 2 - RNA, 22 nt, human
auaaagcuagauaaccgaaagu

SEQ ID NO: 3 - DNA, 26 bp, rat
ggatgtctcgagtgttttctacaaag

SEQ ID NO: 4 - DNA, 25 bp, rat
gaagcagcggccgttgaggctcctc

SEQ ID NO: 5 - DNA, 32 bp, rat
gcatctcgaggtgaccccagggtggttgccac

SEQ ID NO: 6 - DNA, 32 bp, rat
cgatgcggccgcgaatggatcactgttgcaga

(19) United States
(12) Patent Application Publication
(10) Pub. No.: US 2012/0088811 A1
(43) Pub. Date: Apr. 12, 2012

(54) METHOD FOR THE TREATMENT OF ACUTE MYELOID LEUKEMIA

(75) Inventors: Bob Löwenberg, Rotterdam (NL); Mojca Jongen-Lavrencic, Rotterdam (NL); Peter Jacobus Maria Valk, Rotterdam (NL); Su Ming Sun, Rotterdam (NL)

(73) Assignee: ERASMUS UNIVERSITY MEDICAL CENTER ROTTERDAM, ROTTERDAM (NL)

(21) Appl. No.: 13/138,914

(22) PCT Filed: Apr. 22, 2010
(86) PCT No.: PCT/EP2010/055355

§ 371 Date: Dec. 22, 2011

(30) Foreign Application Priority Data
Apr. 22, 2009 (EP) 09158496.1

(51) Int. Cl. A61K 31/7088; A61P 35/02; C07H 21/02

(52) U.S. Cl. 514/44 A; 536/24.5

(57) ABSTRACT

The invention is in the field of molecular medicine and provides methods for the treatment of acute myeloid leukemia. These methods are based on the observation that microRNA-9 and microRNA-9* are involved in the pathogenesis of the disease in that the overexpression of microRNA-9/9* block myeloid differentiation in vitro. More in particular, microRNA-9/9* were found to play a role in leukemic transformation in acute myeloid leukemia.

### FIELD OF THE INVENTION

The invention is in the field of molecular medicine and provides methods for the treatment of acute myeloid leukemia. These methods are based on the observation that microRNA-9 and/or microRNA-9* (microRNA-9/9*) are involved in the pathogenesis of the disease in that the overexpression of microRNA-9/9* block myeloid differentiation in vitro. More in particular, microRNA-9/9* were found to play a role in leukemic transformation in acute myeloid leukemia.

### BACKGROUND OF THE INVENTION

Acute myeloid leukemia (AML) is a remarkably heterogeneous malignant disease, characterized by uncontrolled clonal outgrowth of hematopoietic progenitor cells, blocked at different stages of differentiation. The variable clinical biology and prognosis is mainly determined by somatic cytogenetic abnormalities e.g., t(15;17), t(8;21), inv(16), 11q23, 3q26 abnormalities and monosomal karyotype (1-3) and a variety of gene mutations. Prominent examples are mutations in nucleophosmin-1 (NPM1), fms-like tyrosine kinase receptor (FLT3-interenal tandem duplications (ITD)) and CCAAT binding transcription factor CEBPA (4-7). These acquired genetic aberrations may coexist and are thought to cooperate synergistically in the development of leukemia.

The pathogenesis of AML is a multi-step process affecting cell differentiation, proliferation and apoptosis that ultimately lead to malignant transformation of hematopoietic progenitors. The pathogenetic notion is that at least two, probably in most cases multiple, hits are required for leukemic transformation. One category of genetic abnormalities (class 1) results in constitutive activation of proliferative signaling and involves signaling molecules such as RAS, FLT3 and c-KIT. A second type of lesions (class 2) such as formation of fusion genes, as a result of the chromosomal lesions t(8;21) or inv(16) involving transcription factors result in a block of myeloid differentiation. Various studies in the mice models established the cooperation of both types of genetic deregulation in AML development.

There is a wealth of information on aberrantly expressed genes in AML. Usually, diagnosis of AML and its clinical subgroups is based on the expression of gene profiles collectively referred to as classifiers.

It has recently been demonstrated that microRNA expression profiling in AML also reveals highly distinctive microRNA expression profiles (8).

MicroRNAs are a class of small non-coding RNAs that posttranscriptionally regulate gene expression (9). They regulate gene expression by forming base pairs with sequences in the 3′ untranslated region (3′UTR) of mRNAs thereby repressing translation or inducing degradation of their target mRNA transcripts. To date, more than 400 microRNAs have been described in humans; however, the precise function of these regulatory, non-coding RNAs remains largely obscure.

Differentially expressed microRNAs appeared to characterize particular AML genotypes. Several established oncogenic and tumor suppressor microRNAs, such as microRNA-155, microRNA-21 and let-7 appeared to be associated with specific genetic subtypes of AML(8).

Lu et al. (10) showed that patterns of microRNA gene activity can distinguish between several types of human cancers. They measured the activity of 217 genes encoding microRNA in a new, bead-based flow cytometric microRNA expression profiling method. microRNA signatures may therefore enable classification of cancer. This finding may allow determining the original tissue type which spawned a cancer and targeting a treatment course based on the original tissue type. Lu et al., observed a general downregulation of microRNAs in tumours compared with normal tissues.

Furthermore, they were able to successfully classify poorly differentiated tumours using microRNA expression profiles, whereas messenger RNA profiles were highly inaccurate when applied to the same samples. These findings highlight the potential of microRNA profiling in cancer diagnosis.

Recent studies have shown an association between microRNA expression signatures and (cyto)genetics subsets of AML (8, 11, 12). Furthermore, it was demonstrated that microRNA expression signature containing microRNA-233, -128a -128b and let7b can accurately distinguish AML from acute lymphoblastic leukemia ALL (13).

Increased expression of microRNA-191 and -199a was associated with poor survival in one AML study (12).

A microRNA signature of cytogenetically normal AML was identified that is predictive for adverse prognosis (14).

Another study showed that the let-7/miR-98 cluster is commonly down-regulated whereas microRNA-21, microRNA-155, microRNA-181b, microRNA-221 and microRNA-222 are frequently up-regulated in both solid and hematological tumours (15).

Chen and the colleagues (16) were the first that cloned microRNAs from normal mouse bone marrow and found microRNA-223, -142 and -181 preferentially expressed in hematopoietic tissue. MicroRNA-233 was found to be expressed at low levels in CD34+ hematopoietic progenitors (HPCs) and this expression increased during myeloid differentiation towards granulocytes.

Notwithstanding the fact that theseand other studies provide important information for the diagnosis of AML, they do not provide insights for novel therapies for AML, since it has not been established whether the overexpression or underexpression of a particular gene or microRNA is an epiphenomenon or contributes to the pathogenesis of the disease.

Only a few publications have recently appeared on the relevance of microRNAs in the therapy of cancer. A study of mice altered to produce excess c-myc—a protein implicated in several cancers—shows that miRNA 17-92 polycistron has an effect on the development of cancer. Mice that were engineered to produce a surplus of types of microRNA found in lymphoma cells developed the disease within 50 days and died two weeks later. In contrast, mice without the surplus microRNA lived over 100 days (17). These studies indicate that non-coding RNAs, specifically microRNAs, can modulate tumour formation, and implicate the microRNA-17-92 cluster as a potential human oncogene.

MicroRNA-125b overexpression, that is directly linked to t(2;11) appeared to abrogate myeloid cell differentiation in vitro implying a function of this microRNA in leukemogenesis (18).

Overexpression of microRNA-155 in mouse bone marrow expanded the granulocyte/monocyte population with pathological features characteristic of myeloid neoplasia (19).

In AML, upregulation of microRNA-155 is associated with mutations in the gene of the kinase receptor Fms-like tyrosine kinase-3 (Flt3), so called FLT3-ITD.

In a recent study increased expression of microRNA-196b by MLL fusion proteins has been suggested to contribute to development of AML via increased cell proliferation capacity and survival as well as partial block in differentiation in vitro (20).

Another study found that two types of microRNA (miRNA-17-5p and miRNA-20a) inhibit the E2F1 protein, which regulates cell proliferation.

Johnnidis et al (21), using a loss-of-function allele in mice, demonstrated that the microRNA-223 negatively regulates progenitor proliferation and granulocyte differentiation by targeting Mef2c, a transcription factor that promotes myeloid progenitor proliferation.

Using an acute pro-myelocytic leukemia cell-line model, an auto-regulation model of microRNA-223, CEBPA and NF1A has been proposed (22). Furthermore, PU.1 was found to activate microRNA-223 transcription in mice (23).

Significantly differentially expressed microRNAs during monocyte/macrophage differentiation of the cell line HL60 were identified (24). Besides results obtained from the cell lines models (25, 26) very limited data is available on microRNA expression profiles during normal myeloid differentiation using primary human cells (27).

Functional studies have demonstrated that let-7/miR-98 negatively regulate RAS(28) and v-myc myelocytomatosis viral oncogene homologue (MYC)(29) whereas microRNA-21 negatively regulate programmed cell death 4 (PDCD4)(30) and phosphatase and tensin homologue (PTEN)(31).

Despite of these findings, there remains a need for additional and more specific targets for the treatment of cancers, in particular for the treatment of acute myeloid leukemia (AML).

### SUMMARY OF THE INVENTION

We identified candidate microRNAs with aberrant expression in AML as compared with normal hematopoietic progenitors. MicroRNA-9/9* appears strongly overexpressed in a great number of AML samples and is actively involved in the transformation process.

Moreover, we found that microRNA-9/9* provides an attractive target for the therapy of acute myeloid leukemia. Our experiments show that microRNA-9/9* induces a block in granulocytic differentiation in the murine growth factor dependent myeloid cell line 32D in vitro. This lead us to conclude that acute myeloid leukemia may effectively be treated by interfering with the binding between microRNA-9/9* and their targets.

The invention therefore relates to a method for the treatment of AML wherein a therapeutic composition is administered to a patient in need thereof comprising as an active ingredient a compound capable of interacting with the binding between microRNA-9/9* and its target.

### DETAILED DESCRIPTION OF THE INVENTION

We found that microRNA-9/9* play a role in leukemic transformation in acute myeloid leukemia AML.

As used herein, the term miRNA 9/9* is intended to indicate microRNA 9 and/or microRNA 9*.

MicroRNAs are single-stranded RNAs of 18-25 nucleotides and are generated from precursor molecules. microRNAs are encoded in the genome and transcribed by RNA polymerase II as primary transcripts called pri-microRNAs which are processed in the nucleus into one or more precursor-microRNAs (pre-microRNAs) by the nuclear RNase III, Drosha, and the double-stranded RNA binding protein, Pasha/DGCR8 (32, 33). In the cytoplasm, another RNase III, known as Dicer, further processes the pre-microRNA into double-stranded 23-nucleotide mature microRNA. This microRNA duplex (34) comprises a strand (microRNA strand) which is incorporated into the RNA-induced silencing complex (RISC) and a complementary strand (microRNA* strand), which is usually degraded.

There are several exceptions wherein the mature microRNA* strand is also co-expressed. This is the case with microRNA-9, since also microRNA-9* is usually expressed and is not degraded.

The functional RISC carrying the mature microRNA-9 and/or microRNA-9* can bind to the 3′UTR of their target gene mRNA to result in either mRNA degradation or protein translation inhibition (35, 36)

Genes encoding microRNA-9 and microRNA-9* are located on human chromosomes 1 (miR-9-1), 5 (miR-9-2), and 15 (miR-9-3). Upon processing of these different microRNA-9 precursors, the largely complementary mature microRNA-9 (UCUUUGGUUAUCUAGCUGUAUGA, SEQ ID NO: 1) and microRNA-9* (AUAAAGCUAGAUAACCGAAAGU, SEQ ID NO: 2) are derived.

MicroRNA-9 is enriched in brain and appears important in brain development and lineage commitment (37, 38). In zebrafish, microRNA-9 is important for midbrain-hindbrain boundary definition (39). In mouse cortical development, microRNA-9 appears necessary for appropriate differentiation of Cajal-Retzius cells (40). MicroRNA-9 regulates REST and CoREST and is downregulated in Huntington's Disease (41). microRNA-9 is upregulated in a human neuroblastoma cell line by retinoic acid and is downmodulated in primary neuroblastoma tumors (42). Increased expression of microRNA 9 has been described in AML patients with subtype NPMc+(Ramiro et al., Proc. Natl. Acad. Sci. USA, (2008) 105, 3945-3950) and in subtype 11q23 (Jongen-Lavrencic et al., Blood (2008) 111; 5078-5088).

Increased expression of microRNA-9, which is expressed in beta cells, impairs glucose-stimulated insulin release (43). The latter perturbation of the secretory functions was associated with an increase in the level of granuphilin (SYTL4) a Rab3/Rab27 effector playing a negative modulatory role on insulin exocytosis.

MicroRNA-9 has also been shown to be differentially expressed at successive stages in the course of bronchial carcinogenesis (44). Roccaro el al (45) identified a microRNA signature specific of Waldenstrom Macroglobulinemia with decreased expression of microRNA-9. Recently hypermethylation and decreased expression of microRNA-9 in acute lymphoblastic leukemia (ALL) has been reported correlating with the clinical outcome of these patients (46).

We found that microRNA-9 and microRNA9* are both significantly overexpressed in primary human AML. We performed a supervised analysis using significance analysis of microarray (SAM).

MicroRNA expression of all AML samples was compared to the microRNAs expression patterns of the normal bone marrow CD34+ cells. One of the most discriminating microRNAs were microRNA-9 and microRNA-9*, that both appeared to be more than 100 fold upregulated in AML. This is shown in FIG. 1 for microRNA-9 and in FIGS. 2 and 3 for microRNA 9 and 9*. The results obtained for microRNA-9* were comparable if not identical to the results obtained for microRNA 9.

Furthermore, we found that microRNA-9 and microRNA9* were not or only on a very low level expressed during normal myelopoiesis. We studied the role of microRNA-9 and microRNA9* in hematopoietic differentiation in that we evaluated its expression throughout myeloid development in highly purified cell populations from normal bone marrow and during in vitro myeloid differentiation assay of normal human CD34+ cell.

Using both approaches the expression of microRNA-9 and microRNA-9* was at the detection level of the quantitative RT-PCR assay in myeloid progenitors and remained so as granulocytic differentiation proceeds through promyelocytes, metamyelocytes to mature neutrophils. This is shown in FIG. 4.

These data show that microRNA-9 and microRNA-9* do not have a function in normal myeloid differentiation. We conclude that the aberrant expression of microRNA-9/9*in AML as detailed above is associated with leukemogenesis rather than that it reflects the differentiation stage of AML. In addition, we found that miRNA-9/9* induces a block in myeloid differentiation of 32D cells (murine myeloid leukemia cell line) and primary murine bone marrow cells in vitro. For that purpose, microRNA-9/9* was retrovirally transduced into the 32D cell line. This cell line is known to proliferate when interleukin-3 (IL-3) is added to the medium but is able to differentiate towards neutrophils when IL3 is replaced by granulocyte-colony stimulating factor (G-CSF). Using hematocytology (cytospins) cell staining of 32D cells we observed a block in the differentiation under IL-3 conditions and persistent cell proliferation under the G-CSF condition (FIG. 5). Furthermore, microRNA-9 and microRNA-9* were also introduced into primary murine bone marrow cells. microRNA-9/9* overexpression resulted in decreased numbers of mature neutrophils (CD11b+Gr1+ cells) in vitro as defined by flow-cytometry. This corroborates our findings in the 32D cell line.

Cytospin stainings were performed on various time points (day 0, 3, 7, 10, 15) during the differentiation assay with G-CSF. 32D cells transduced with control vector were differentiating from blasts on day 0 to completely mature granulocytes on day 7. Interestingly, in 32D cells transduced with microRNA-9/9* we observed block in differentiation, since no mature granulocytes were observed on day 7 and all later time points.

Furthermore, microRNA-9/9* was introduced to the primary murine bone marrow cells. microRNA-9/9* overexpression using retroviral vector resulted in decreased percentage of mature neutrophils (CD11b+Gr1+ cells) in vitro examined by immunophenotyping.

We were able to inhibit the proliferatin of primary human AML cells by contacting them with miRNA 9* inhibitor. This is shown in FIGS. 6 to 11. A LNA (locked nucleic acid) based antisense inhibitor against miR-9* or a control inhibitor was introduced into fresh isolated AML blast cells by transfection using a concentration of 150 mM. The proliferation of the AML wildtype blast cells or cells transfected with control- and miR-9*-inhibitor was measured under various cytokine stimulation conditions using [3H] Thymidine incorporation assay in triplicate. Two independent AML samples obtained from different patients showed a decrease in cell proliferation upon treatment with inhibitor against miR-9* as compared with control inhibitor and wildtype control cells.

In summary, we found that microRNA-9/9* overexpression results in functional phenotype, namely block in myeloid differentiation. Based on these results we conclude that microRNA-9/9* plays a role in leukemic transformation in AML. We were also able to show that microRNA-9/9* inhibitors indeed effectively blocked the proliferation of AML blast cells. Therewith these inhibitors may prove effective therapeutic compounds for inhibiting cell proliferation in AML, in other words, compounds that interfere with the binding of microRNA-9 or microRNA-9* to their targets may be useful in the treatment of AML.

The findings as detailed above make it possible to intervene with the interaction of microRNA-9/9* and their cellular targets, thereby providing a method for the treatment of AML. The invention therefore provides a method for the treatment of AML wherein a therapeutic composition is administered to a patient in need thereof comprising as an active ingredient a compound capable of interacting with the binding between microRNA-9 or 9* and their targets.

In the alternative, the intervention may be at the level of microRNA-9/9* transcription and/or processing so that the level of mature microRNA-9/9* in the cell is decreased. Hence, the invention also relates to a method for the treatment of AML wherein a therapeutic composition is administered to a patient in need thereof comprising as an active ingredient a compound capable of decreasing the cellular level of microRNA-9/9*, preferably of mature microRNA-9 or 9*.

This may be effected by providing a compound that interferes with the transcription or processing of precursor microRNA-9 9*. The invention therefore preferably relates to a method as described above wherein the compound is capable of interfering with the transcription or processing of precursor microRNA-9/9*.

Compounds, which are capable of interfering with the interaction of microRNA-9 and/or microRNA-9* and their targets are known in the art. They may come in two categories; first the class of compounds that interfere with the binding by blocking the binding site or the target and making it inaccessible for microRNA-9/9* and second the class of compounds that bind to microRNA-9/9* to prevent them from binding to their targets.

The first class of compounds may be developed such that they are specific for each target. This is a routine procedure for the skilled person since the sequences of the RNA targets of microRNA-9/9* are known and complementary nucleotides may easily be developed.

In order to develop such compounds, the skilled person should be aware of the principles of microRNA-target recognition. These are detailed in Brennecke et al., PLOS Biology, (2005) 3(3) e85; 0404-0418. Several web-based in silico algorithms are also available (www.targetscan.org; www.pictart.org; www.ebi.ac.uk/enright-srv/microcosm/htdocs/targets/v5/predicting) for the prediction of potential microRNA targets based on miRNA-seed and conservation. It is important to note in this respect that microRNA-9 and microRNA-9* regulate a completely different set of targets since they are not sharing the same seed sequence.

The second category of compounds that are capable of interfering with the interaction of microRNA-9 and/or microRNA-9* and their targets are the class of compounds that bind to microRNA-9 or 9* and prevent their binding to any microRNA-9 or 9* target.

In a preferred embodiment, compounds capable of interfering with the interaction of microRNA-9 and/or microRNA-9* and their targets are compounds comprising a nucleotide sequence essentially complementary to the sequence of mature microRNA-9 or 9*, more in particular the sequences shown in SEQ ID NO: 1 and SEQ ID NO: 2. Preferably, such compounds are essentially complementary to at least 6 consecutive nucleotides, such as 7, 8, 9, 10, 11, 12, 13, 14, 15 or more nucleotides. Such compounds effectively hybridize to microRNA-9/9* under physiological conditions in order to prevent microRNA-9/9* from binding to their targets. Preferably, the compound hybridizes to the whole of microRNA-9 or microRNA-9*.

Such compounds are known in the art and are even commercially available. Well known examples of such compounds are called antagomirs (Exiqon) and antimirs (Dharmacon).

The seed sequences of microRNA-9/9* are important for target recognition. The seed sequence consists of at least 6 nucleotides starting at the position 2 at the 5′ of of the mature microRNA. It may extend further in the 3′ direction but is usually considered to comprise not more than 8 nucleotides. The seed region of mature microRNA-9 therefore comprises the nucleotide sequence CUUUGG, CUUUGGU or CUUUGGUU. The seed region of mature microRNA-9* comprises the nucleotide sequence UAAAGC, UAAAGCU or UAAAGCUA.

The invention therefore preferably relates to compounds capable of interacting with the seed sequence of microRNA-9 or microRNA-9*. Preferably, such compounds are capable of hybridizing to the seed sequences under physiological conditions. Compounds which have a nucleotide sequence essentially complementary to the seed sequence of microRNA-9/9* are preferred.

Such compounds and additional compounds may easily be found by using known techniques, for instance a Renilla Luciferase assay wherein microRNA-9 or 9* is allowed to bind to 3′ UTR of candidate wild type and mutated target. Additional compounds useful in the invention may also be found by immunoblotting (Western assay) wherein protein levels of candidate microRNA9/9* targets can be examined and compared in cells with or without overexpression of microRNA9/9* in combination with inhibitory compound. The invention therefore also relates to a compound capable of interacting with the binding between microRNA-9/9* and their targets for the treatment of AML. Also, the invention relates to a compound capable of decreasing the cellular level of microRNA-9/9* for the treatment of AML.

In summary, the invention relates to a compound capable of interacting with the binding between microRNA-9 and/or microRNA-9* and their targets for the treatment of AML. More in particular, the invention relates to a compound for the treatment of AML wherein the compound is capable of binding to microRNA-9/9*, more in particular a compound comprising a nucleotide sequence capable of binding to microRNA-9 and/or microRNA-9*. The nucleotide sequence may be a sequence comprising a protein nucleid acid (PNA) or a locked nucleic acid (LNA) or any other form of nucleic acid or nucleic acid binding sequence.

In other words, the invention relates to a method for the treatment of AML wherein a compound capable of interfering with the binding of microRNA-9/9* to its target is administered to a patient in need of such a treatment

In a preferred embodiment, the compound comprises a nucleotide sequence essentially complementary to the sequence of mature microRNA-9 or microRNA-9*. Such a compound may consist of a strain of nucleotides of at least 6, preferably 7, 8, 9 or 10 nucleotides. Longer stretches of nucleotides are also feasible.

Preferably, the compound binds to the seed sequence. This may be accomplished by a sequence that hybridizes to the seed sequence, i.e. it comprises a sequence essentially complementary to at least 6 nucleotides of the sequence of microRNA-9 and/or microRNA-9* starting from the second nucleotide at the 5′ end of mature microRNA. Such compounds exhibited very good binding properties to the microRNAs and are therefore preferred, much like compounds comprising 7, 8, 9, 10, or more nucleotides in common with microRNA-9 and/or microRNA-9*.

The term “essentially complementary” in this respect means that the sequences exhibit more than 60% sequence homology, such as 70, 80, 85, 90 or more than 90 such as 95% or even 100%.

Mature microRNA-9 has the following nucleotide sequence: UCUUUGGUUAUCUAGCUGUAUGA, (SEQ ID NO: 1) and mature microRNA-9* has the nucleotide sequence AUAAAGCUAGAUAACCGAAAGU (SEQ ID NO: 2).

In a further preferred embodiment, the compound comprises a sequence which is essentially complementary to the whole sequence of microRNA-9 and/or microRNA-9*.

Sometimes it is preferred to treat only those patients that may benefit most from a treatment or a compound according to the invention. In that respect, preferably patients with an AML characterized by increased expression levels of microRNA-9 or microRNA-9* are treated. Preferably, those patients from AML subgroubs that are characterized by NPM1 mutation or 11q23 abnormalities since those patients show the highest up-regulation of microRNA-9 and microRNA-9* within the heterogeneous AML cohorts. Subgroups of AML patient with t(8;21), -5/-7 and CEBPA mutations may be somewhat less likely candidates for such a treatment since in these patients microRNA-9/9* were down-regulated.as compared to the rest of AMLs (Jongen-Lavrencic et al., Blood 2008).

The skilled person is aware of methods (cytogenetic analysis and molecular diagnostics) on how to distinguish the relevant subgroups of AML. Elevated or increased levels of microRNA-9/9* in this respect refer to levels of the microRNAs which are above the normal level, i.e. the level of microRNA-9/9* in a population of normal individuals without AML.

### LEGENDS TO THE FIGURES

FIG. 1: MicroRNA-9 is Significantly Overexpressed in Primary Human AML

SDS 2.3 software (Applied Biosystems) was used to analyze microRNA-9 expression data obtained by Real-time RT-PCR. The geometric mean of several snRNAs (small nuclear RNAs) expression was used for internal normalization. The comparative relative quantification method, 2-ddCt was used to calculate the relative expression (fold change) of the microRNA-9 in AMLs compared to normal CD34+ cells. Relative expression (fold change) of microRNA-9 is presented on the vertical axis for all 215 AML samples in the cohort (horizontal axis).

FIG. 2: MicroRNA-9 is Significantly Overexpressed in Primary Human AML

SDS 2.3 software (Applied Biosystem) was used to analyze microRNA-9 expression data obtained by Real-time RT-PCR. The expression of RNU48 was used for internal normalization. The expression relative to the internal controls represented by -dCt values is presented on the vertical axis for the different granulocytic maturation stages for all 215 AML samples in the cohort (horizontal axis).

FIG. 3: MicroRNA9* is Significantly Overexpressed in Primary Human AML

SDS 2.3 software (applied Biosystem) was used to analyze microRNA-9* expression data obtained by Real-time RT-PCR. The geometric mean of RNU24 and RNU66 expression was used for internal normalization. The expression relative to the internal controls of miR-9* represented by -dCt values is presented on the vertical axis for all 215 AML patients in the cohort (horizontal axis).

FIG. 4: MicroRNA-9/9* Expression in Normal Human Granulopoiesis

SDS 2.3 software (Applied Biosystem) was used to analyze microRNA-9 (and miR-9* expression data obtained by Real-time RT-PCR. The expression of RNU48 was used for internal normalization. The expression relative to the internal controls represented by -dCt values is presented on the vertical axis for the different granulocytic maturation stages.

FIG. 5: Overexpression of MicroRNA-9/9* in 32D Cell Line Leads to Block in Myeloid Differentiation.

MicroRNA-9/9* or control vector were transduced into the murine myeloid leukemia cell line (32D) using retroviral vector containing GFP (green florescence protein) reporter. Cells with high GFP expression were sorted and used in a proliferation/differentiation assay. Overexpression of microRNA-9/9* had no effect on proliferating capacity of 32D cells growing in IL-3 supplemented medium. However, when growing them in G-CSF containing medium, microRNA-9/9* transduced cells continue with proliferation while cells transduced with control vector decreased in grow.

FIGS. 6-11: Negative Effect of MicroRNA-9* Inhibitor on Proliferation of Primary Human AML Cells.

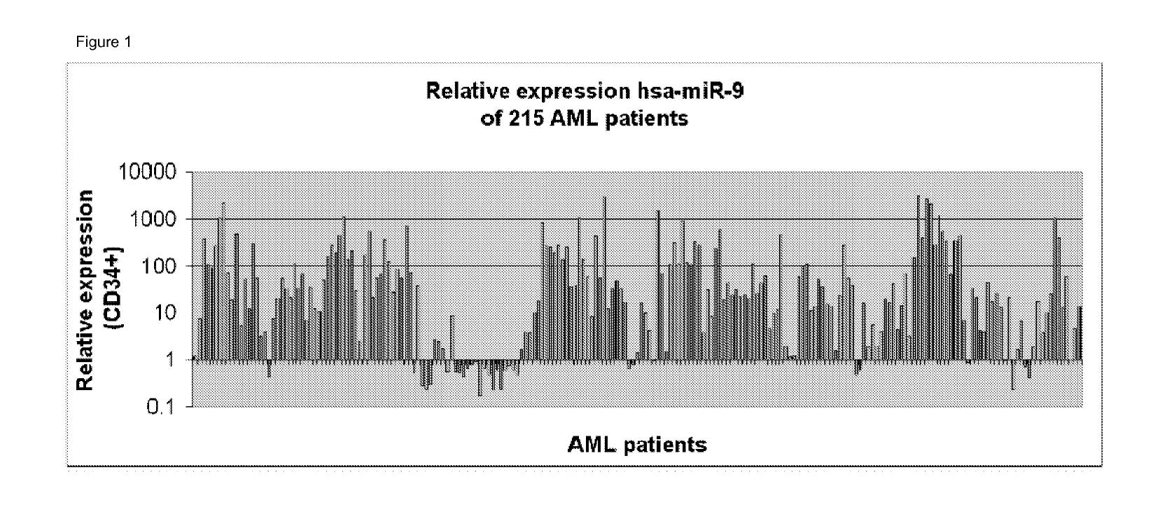

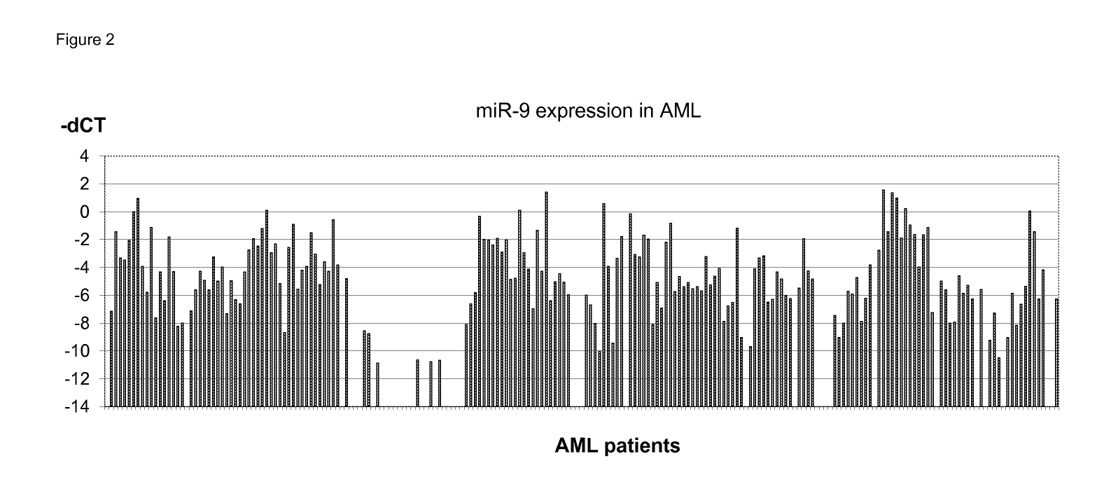

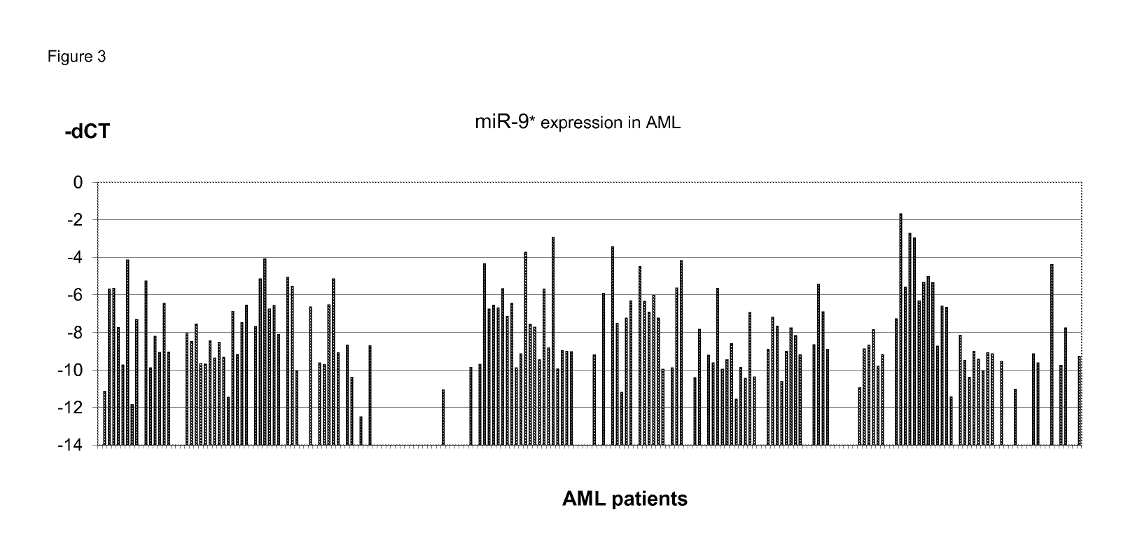

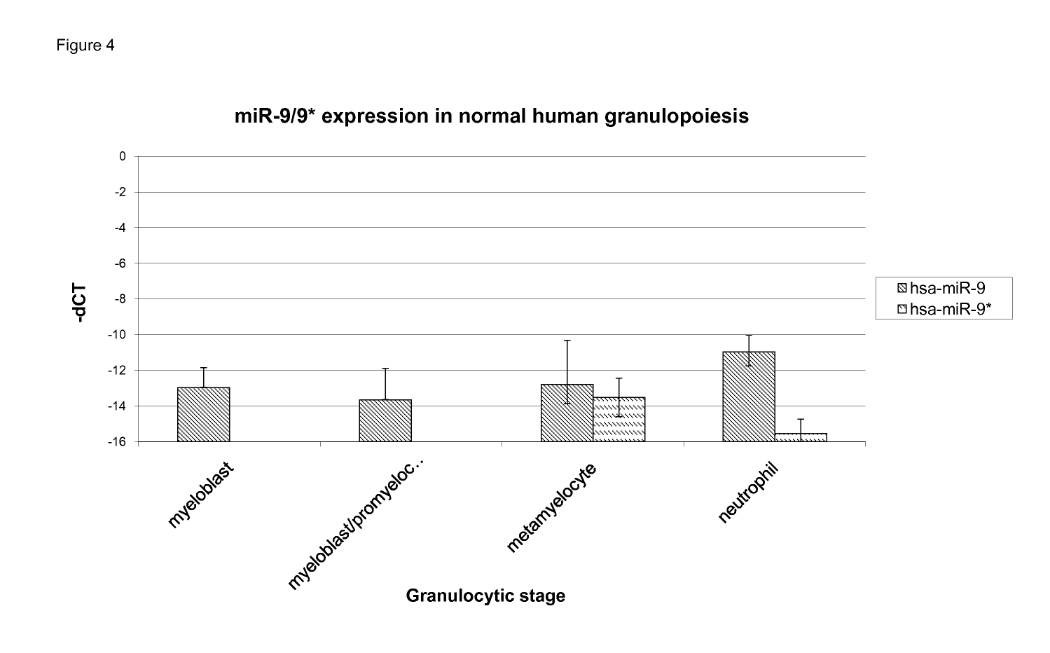

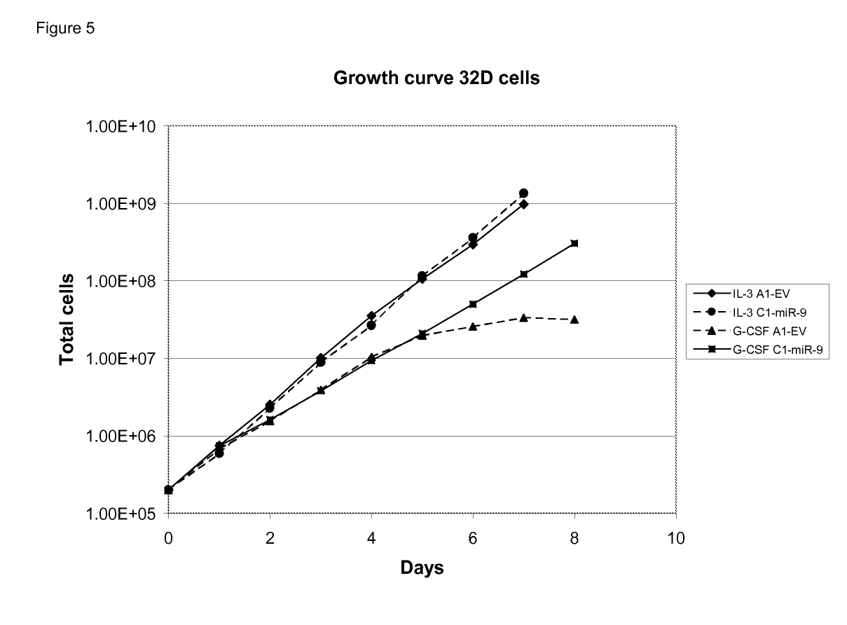

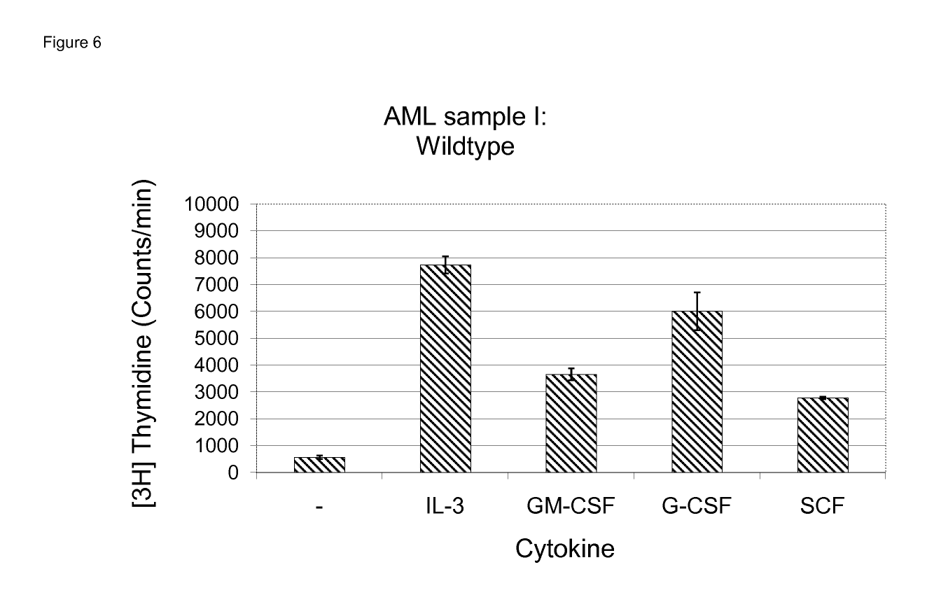

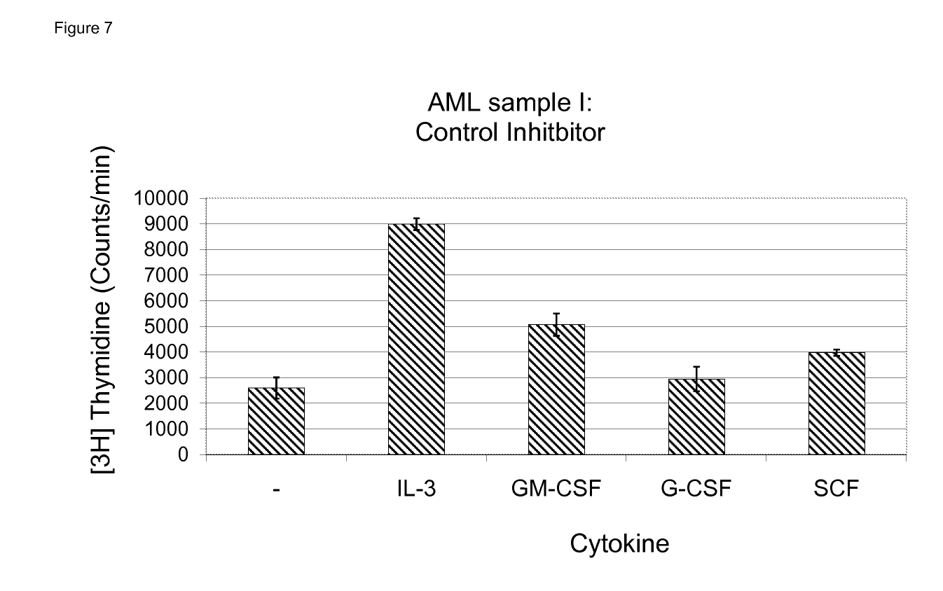

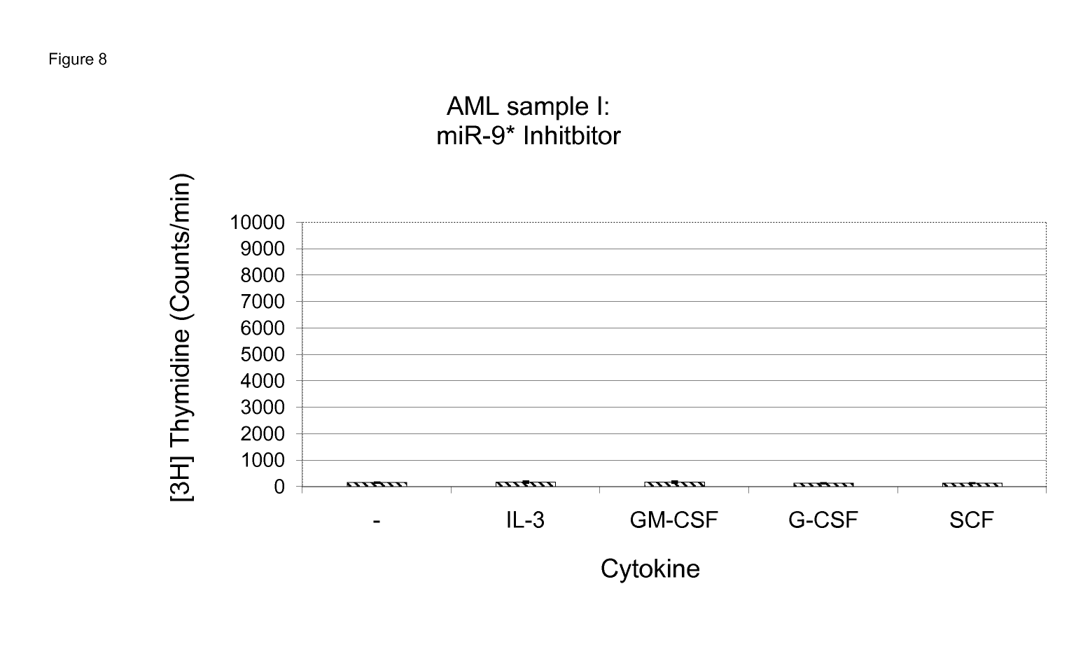

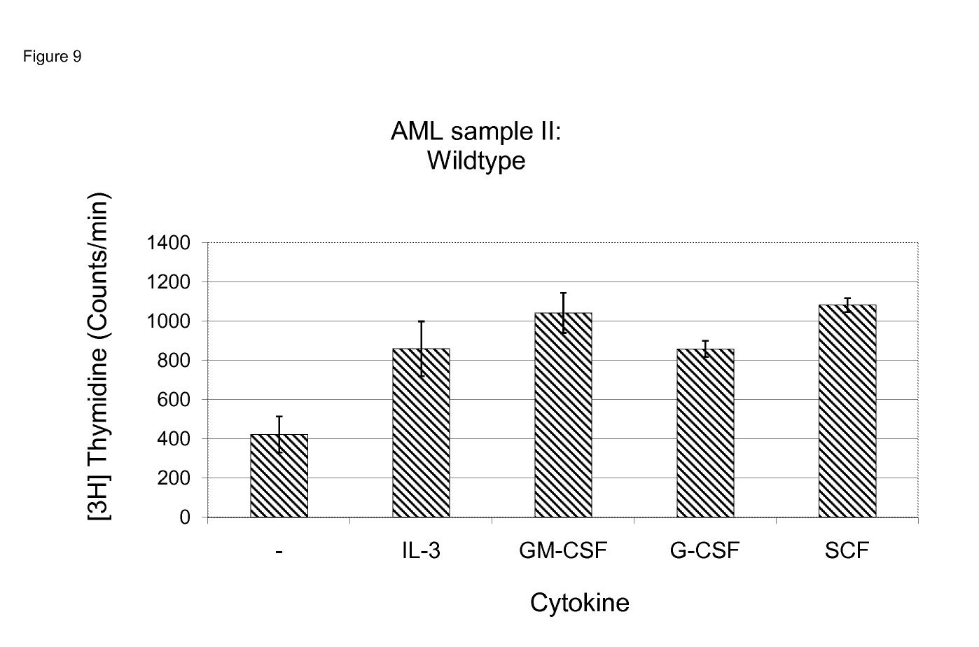

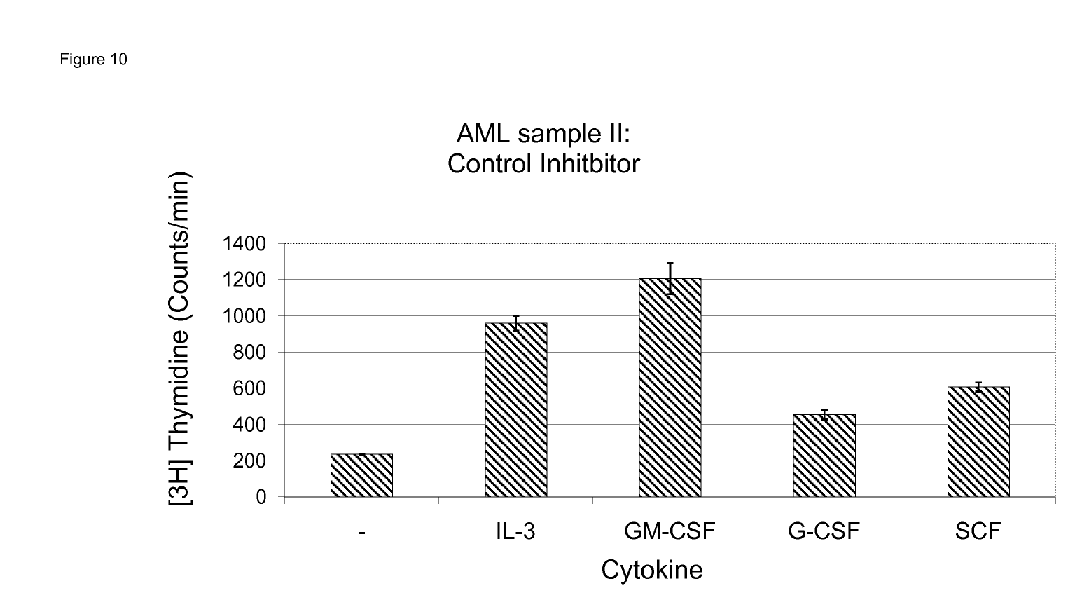

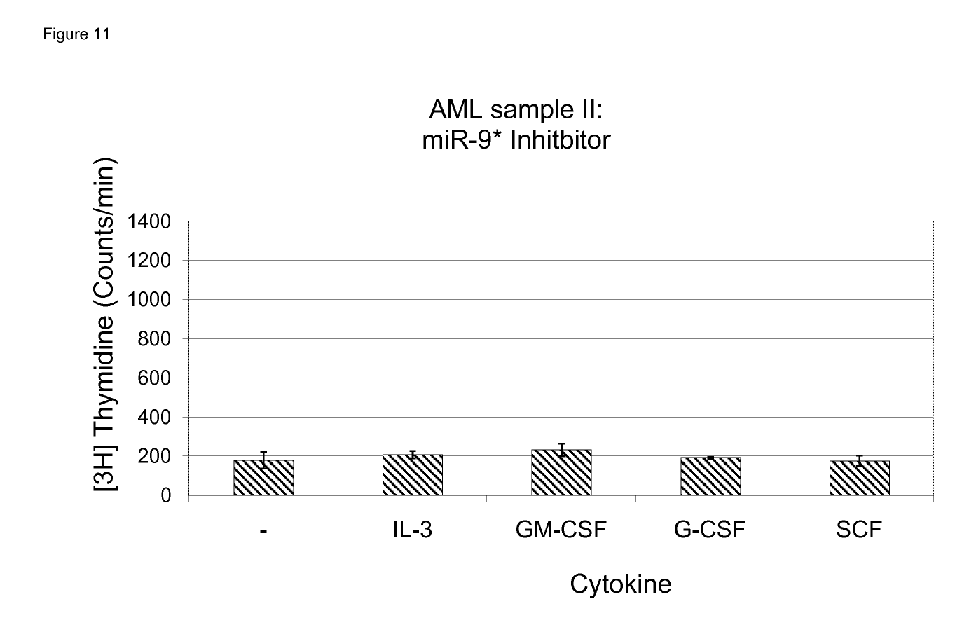

A LNA (locked nucleic acid) based antisense inhibitor against miR-9* or a control inhibitor was introduced into fresh isolated AML blast cells by transfection using a concentration of 150 mM. The proliferation of the AML wildtype blast cells or cells transfected with control- and miR-9*-inhibitor was measured under various cytokine stimulation conditions using [3H] Thymidine incorporation assay in triplicate. Two independent samples from different AML patients (sample I and II) showed a decrease in cell proliferation upon treatment with inhibitor against miR-9* as compared with control inhibitor and wildtype control cells.

### EXAMPLES

### Example 1

### Testing the Activity of New Potential MicroENA-9 Inhibitory Compounds Using a Known microRNA-9 Target Onecut2 (OC2)

Luciferase assay: Onecut2 (OC2) transcription factor was previously identified as a target of microRNA-9 (43). To generate the 3′-UTR-0C2-luc construct, the 3′-UTR segment of the rat oc2 gene was amplified by PCR from genomic DNA and inserted in the multiple cloning site of the psiCHECK-1 plasmid (Promega, Madison, Wis.). The multiple cloning site of this plasmid is located in the 3′-UTR of the Renilla luciferase gene between the stop codon and an artificial polyadenylation site. The sequences of the PCR primers were as follows: sense, 5′-GGATGTCTCGAGTGTTTTCTACAAAG-3′, (SEQ ID NO: 3) and antisense, 5′-GAAGCAGCGGCCGTTGAGGCTCCTC-3′ (SEQ ID NO: 4). The microRNA-9-sensor construct can be obtained by cloning the microRNA-9 mature sequence in the antisense orientation in the 3′-UTR of the Renilla luciferase gene of psiCHECK-1 (Promega).

Transient transfections of plasmids are than performed using the Effectene transfection kit (Qiagen, Valencia, Calif.). Experiments involving transient transfections of microRNA-9 and control microRNA can be carried out with Lipofectamine 2000 (Invitrogen) using 100 nM RNA duplexes. Luciferase activities is measured 2 days after transfection with the dual-luciferase reporter assay system (Promega). For 3′-UTR-OC2-luc and microRNA-9-sensor co-transfections with the Firefly luciferase SV40 pGL3 promoter (Promega) or with the Renilla luciferase pRLCMV (pRLrenilla), respectively, can be used for normalization.

### Example 2

### Testing the Activity of New Potential MicroRNA-9* Inhibitory Compounds Using the Known microrna-9* Targets REST and CoREST

Luciferase assay: Target CoRESTwas previously identified as a target of microRNA-9* (Packer et al., J. neurosci. 2008 (53) 14341-6). To generate the 3′-UTR-CoREST-luc construct, the 3′-UTR segment of the gene was be amplified by PCR from genomic DNA and inserted in the multiple cloning site of the psiCHECK-1 plasmid (Promega, Madison, Wis.). The multiple cloning site of this plasmid is located in the 3′-UTR of the Renilla luciferase gene between the stop codon and an artificial polyadenylation site. The sequences of the PCR primers are as follows: sense, CoREST 3′ UTR 5′-GCATCTCGAGGTGACCCCAGGGTGGTTGCCAC-3′ (SEQ ID NO: 5). and 5′-CGATGCGGCCGCGAATGGATCACTGTTGCAGA-3′.(SEQ ID NO: 6)

The microRNA-9*-sensor construct can be obtained by cloning the microRNA-9* mature sequence in the antisense orientation in the 3′-UTR of the Renilla luciferase gene of psiCHECK-1 (Promega).

Transient transfections of plasmids are than performed using the Effectene transfection kit (Qiagen, Valencia, Calif.). Experiments involving transient transfections of microRNA-9* and control microRNA can be carried out with Lipofectamine 2000 (Invitrogen) using 100 nM RNA duplexes. Luciferase activities is measured 2 days after transfection with the dual-luciferase reporter assay system (Promega). For 3′-UTR-coREST-luc and microRNA-9*-sensor co-transfections with the Firefly luciferase SV40 pGL3 promoter (Promega) or with the Renilla luciferase pRLCMV (pRLrenilla), respectively, can be used for normalization.

### Example 3

### Immunoblot Analysis

For Western blot analysis, the cells must be washed once in icecold phosphate-buffered saline, protein extracts are prepared as described before (43), 15 μg of proteins is subjected to SDS-PAGE and transferred on polyvinylidene difluoride membranes. The membranes are blocked for 60 min in a buffer containing 0.1% Tween 20 and 5% milk and were then incubated overnight at 4° C. with a primary antibody raised against rat 002 (amino acids 36-311) (32). Immunoreactive bands must be visualized using a chemiluminescent substrate (Amersham Biosciences) after incubation of the membrane for 60 min with secondary antibody conjugated to horseradish peroxidase.

### REFERENCES

1. Slovak M L, Kopecky K J, Cassileth P A, Harrington D H, Theil K S, Mohamed A, Paietta E, Willman C L, Head D R, Rowe J M, et al. (2000) Blood96, 4075-4083.
2. Grimwade D, Walker H, Harrison G, Oliver F, Chatters S, Harrison C J, Wheatley K, Burnett A K, & Goldstone A H (2001) Blood98, 1312-1320.
3. Breems D A, Van Putten W L, De Greef G E, Van Zelderen-Bhola S L, Gerssen-Schoorl K B, Mellink C H, Nieuwint A, Jotterand M, Hagemeijer A, Beverloo H B, et al. (2008) J Clin Oncol 26, 4791-4797.
4. Dohner K, Schlenk R F, Habdank M, Scholl C, Rucker F G, Corbacioglu A, Bullinger L, Frohling S, & Dohner H (2005) Blood106, 3740-3746.
5. Frohling S, Schlenk R F, Breitruck J, Benner A, Kreitmeier S, Tobis K, Dohner H, & Dohner K (2002) Blood100, 4372-4380.
6. Barjesteh van Waalwijk van Doorn-Khosrovani S, Erpelinck C, Meijer J, van Oosterhoud S, van Putten W L, Valk P J, Berna Beverloo H, Tenen D G, Lowenberg B, & Delwel R (2003) Hematol J 4, 31-40.
7. Falini B, Nicoletti I, Martelli M F, & Mecucci C (2007) Blood109, 874-885.
8. Jongen-Lavrencic M, Sun S M, Dijkstra M K, Valk P J, & Lowenberg B (2008) Blood 111, 5078-5085.
9. Bartel D P (2004) Cell 116, 281-297.
10. Lu J, Getz G, Miska E A, Alvarez-Saavedra E, Lamb J, Peck D, Sweet-Cordero A, Ebert B L, Mak R H, Ferrando A A, et al. (2005) Nature 435, 834-838.
11. Li Z, Lu J, Sun M, Mi S, Zhang H, Luo R T, Chen P, Wang Y, Yan M, Qian Z, et al. (2008) Proc Natl Acad Sci USA 105, 15535-15540.
12. Garzon R, Volinia S, Liu C G, Fernandez-Cymering C, Palumbo T, Pichiorri F, Fabbri M, Coombes K, Alder H, Nakamura T, et al. (2008) Blood111, 3183-3189.
13. Mi S, Lu J, Sun M, Li Z, Zhang H, Neilly M B, Wang Y, Qian Z, Jin J, Zhang Y, et al. (2007) Proc Natl Acad Sci USA 104, 19971-19976.
14. Marcucci G, Radmacher M D, Maharry K, Mrozek K, Ruppert A S, Paschka P, Vukosavljevic T, Whitman S P, Baldus C D, Langer C, et al. (2008) N Engl J Med 358, 1919-1928.
15. Volinia S, Calin G A, Liu C G, Ambs S, Cimmino A, Petrocca F, Visone R, Iorio M, Roldo C, Ferracin M, et al. (2006) Proc Natl Acad Sci USA 103, 2257-2261.
16. Chen C Z, Li L, Lodish H F, & Bartel D P (2004) Science 303, 83-86.
17. He L, Thomson J M, Hemann M T, Hernando-Monge E, Mu D, Goodson S, Powers S, Cordon-Cardo C, Lowe S W, Hannon G J, et al. (2005) Nature 435, 828-833.
18. Bousquet M, Quelen C, Rosati R, Mansat-De Mas V, La Starza R, Bastard C, Lippert E, Talmant P, Lafage-Pochitaloff M, Leroux D, et al. (2008) J Exp Med 205, 2499-2506.
19. O'Connell R M, Rao D S, Chaudhuri A A, Boldin M P, Taganov K D, Nicoll J, Paquette R L, & Baltimore D (2008) J Exp Med 205, 585-594.
20. Popovic R, Riesbeck L E, Velu C S, Chaubey A, Zhang J, Achille N J, Erfurth F E, Eaton K, Lu J, Grimes H L, et al. (2009) Blood.
21. Johnnidis J B, Harris M H, Wheeler R T, Stehling-Sun S, Lam M H, Kirak O, Brummelkamp T R, Fleming M D, & Camargo F D (2008) Nature 451, 1125-1129.
22. Fazi F, Rosa A, Fatica A, Gelmetti V, De Marchis M L, Nervi C, & Bozzoni I (2005) Cell 123, 819-831.
23. Fukao T, Fukuda Y, Kiga K, Sharif J, Hino K, Enomoto Y, Kawamura A, Nakamura K, Takeuchi T, & Tanabe M (2007) Cell 129, 617-631.
24. Fontana L, Pelosi E, Greco P, Racanicchi S, Testa U, Liuzzi F, Croce C M, Brunetti E, Grignani F, & Peschle C (2007) Nat Cell Biol 9, 775-787.
25. Shi B, Prisco M, Calin G, Liu C G, Russo G, Giordano A, & Baserga R (2006) J Cell Physiol 207, 706-710.
26. Garzon R, Pichiorri F, Palumbo T, Visentini M, Aqeilan R, Cimmino A, Wang H, Sun H, Volinia S, Alder H, et al. (2007) Oncogene 26, 4148-4157.
27. Georgantas R W, 3rd, Hildreth R, Morisot S, Alder J, Liu C G, Heimfeld S, Calin G A, Croce C M, & Civin C I (2007) Proc Natl Acad Sci USA 104, 2750-2755.
28. Johnson S M, Grosshans H, Shingara J, Byrom M, Jarvis R, Cheng A, Labourier E, Reinert K L, Brown D, & Slack F J (2005) Cell 120, 635-647.
29. Sampson V B, Rong N H, Han J, Yang Q, Aris V, Soteropoulos P, Petrelli N J, Dunn S P, & Krueger L J (2007) Cancer Res67, 9762-9770.
30. Lu Z, Liu M, Stribinskis V, Klinge C M, Ramos K S, Colburn N H, & Li Y (2008) Oncogene 27, 4373-4379.
31. Meng F, Henson R, Wehbe-Janek H, Ghoshal K, Jacob S T, & Patel T (2007) Gastroenterology 133, 647-658.
32. Saini H K, Griffiths-Jones S, & Enright A J (2007) Proc Natl Acad Sci USA 104, 17719-17724.
33. Gregory R I, Yan K P, Amuthan G, Chendrimada T, Doratotaj B, Cooch N, & Shiekhattar R (2004) Nature 432, 235-240.
34. Ketting R F, Fischer S E, Bernstein E, Sijen T, Hannon G J, & Plasterk R H (2001) Genes Dev 15, 2654-2659.
35. Okamura K, Ishizuka A, Siomi H, & Siomi M C (2004) Genes Dev 18, 1655-1666.
36. Liu J, Carmell M A, Rivas F V, Marsden C G, Thomson J M, Song J J, Hammond S M, Joshua-Tor L, & Hannon G J (2004) Science 305, 1437-1441.
37. Krichevsky A M, Sonntag K C, Isacson O, & Kosik K S (2006) Stem Cells 24, 857-864.
38. Sempere L F, Freemantle S, Pitha-Rowe I, Moss E, Dmitrovsky E, & Ambros V (2004) Genome Biol 5, R13.
39. Leucht C, Stigloher C, Wizenmann A, Klafke R, Folchert A, & Bally-Cuif L (2008) Nat Neurosci 11, 641-648.
40. Shibata M, Kurokawa D, Nakao H, Ohmura T, & Aizawa S (2008) J Neurosci 28, 10415-10421.
41. Packer A N, Xing Y, Harper S Q, Jones L, & Davidson B L (2008) J Neurosci 28, 14341-14346.
42. Laneve P, Di Marcotullio L, Gioia U, Fiori M E, Ferretti E, Gulino A, Bozzoni I, & Caffarelli E (2007) Proc Natl Acad Sci USA 104, 7957-7962.
43. Plaisance V, Abderrahmani A, Perret-Menoud V, Jacquemin P, Lemaigre F, & Regazzi R (2006) J Biol Chem 281, 26932-26942.
44. Mascaux C, Laes J F, Anthoine G, Haller A, Ninane V, Burny A, & Sculier JP (2009) Eur Respir J 33, 352-359.
45. Roccaro A M, Sacco A, Chen C, Runnels J, Leleu X, Azab F, Azab A K, Jia X, Ngo H T, Melhem M R, et al. (2008) Blood.
46. Roman-Gomez J, Agirre X, Jimenez-Velasco A, Arqueros V, Vilas-Zornoza A, Rodriguez-Otero P, Martin-Subero I, Garate L, Cordeu L, San Jose-Eneriz E, et al. (2009) J Clin Oncol 27, 1316-1322.

1. A method of treating a subject with Acute Myeloid Leukemia (AML), the method comprising:
administering to the subject a molecule able to interfere with the binding of microRNA-9 and/or microRNA-9* to its respective target.

2. The method according to claim 1, wherein the molecule comprises a nucleotide sequence able to bind to microRNA-9 and/or microRNA-9*.

3. The method according to claim 1, wherein the molecule comprises a nucleotide sequence essentially complementary to at least 6 consecutive nucleotides of the sequence of mature microRNA-9 and/or microRNA-9*.

4. The method according to claim 1, wherein the molecule comprises a nucleotide sequence that is essentially complementary to the seed sequence of microRNA-9 and/or microRNA-9*.

5. The method according to claim 1, wherein the molecule comprises a nucleotide sequence able to hybridize to mature microRNA-9/9* under physiological conditions.

6. A method of treating a subject with Acute Myeloid Leukemia (AML), the method comprising:
administering to the subject a molecule able to decrease the cellular level of mature microRNA-9 and/or microRNA-9*.

7. A method of treating a subject with Acute Myeloid Leukemia (AML), the method comprising:
administering to the subject a molecule able to interfere with the binding of microRNA-9 and/or microRNA-9* to its target., wherein the AML is characterized by increased expression levels of microRNA-9 or microRNA-9*.

8. The method according to claim 7 wherein the AML is characterized by a NPM1 mutation or an 11q23 chromosomal abnormality.

9. (canceled)

10. (canceled)

11. The method according to claim 6, wherein the AML is characterized by increased expression levels of microRNA-9 or microRNA-9*.

12. The method according to claim 11 wherein the AML is characterized by a NPM1 mutation or an 11q23 chromosomal abnormality.

13. A molecule able to interfere with the binding of microRNA-9 and/or microRNA-9* to its respective target.

14. The molecule of claim 13, wherein the molecule comprises a nucleotide sequence able to bind to microRNA-9 and/or microRNA-9*.

15. The molecule of claim 13, wherein the molecule comprises a nucleotide sequence essentially complementary to at least 6 consecutive nucleotides of the sequence of mature microRNA-9 and/or microRNA-9*.

16. The molecule of claim 13, wherein the molecule comprises a nucleotide sequence that is essentially complementary to the seed sequence of microRNA-9 and/or microRNA-9*.

17. The molecule of claim 13, wherein the molecule comprises a nucleotide sequence able to hybridize to mature microRNA-9/9* under physiological conditions.

18. The molecule of claim 13, wherein the molecule is able to decrease the cellular level of mature microRNA-9 and/or microRNA-9*.
